(19) United States
(12) Patent Application Publication
(10) Pub. No.: US 2025/0388717 A1
(43) Pub. Date: Dec. 25, 2025

(54) POLYUREA ACRYLATE OLIGOMER CONTAINING PROTECTED GROUPS AND PREPARATION METHOD AND APPLICATION THEREOF

(71) Applicant: Zhejiang University, Hangzhou (CN)

(72) Inventors: Zizheng FANG, Hangzhou (CN); Tao XIE, Hangzhou (CN); Jingjun WU, Hangzhou (CN); Qian ZHAO, Hangzhou (CN)

(21) Appl. No.: 18/812,026

(22) Filed: Aug. 22, 2024

(30) Foreign Application Priority Data
Jun. 25, 2024 (CN) 202410823831.2

(51) Int. Cl. C08G 18/67; C08G 18/12

(52) U.S. Cl. CPC C08G 18/672; C08G 18/12

(57) ABSTRACT

Disclosed are a polyurea acrylate oligomer containing protected groups X3 and a preparation method and application thereof, belonging to the technical field of polymer materials, the present disclosure introduces a protected group into a polyurea acrylate oligomer, where diisocyanate, diols containing X₃ groups and common diols are mixed, and reacted at a temperature ranging from room temperature to 80 degrees Celsius for 1-6 hours to generate polyurethane prepolymer; the polyurethane prepolymer is mixed with a hindered amine acrylate and reacted at 50 to 100 degrees Celsius for 1 to 6 hours to generate the polyurea acrylate oligomer containing protected groups.

### CROSS-REFERENCE TO RELATED APPLICATIONS

This application claims priority to Chinese Patent Application No. 202410823831.2, filed on Jun. 25, 2024, the contents of which are hereby incorporated by reference.

### TECHNICAL FIELD

The present disclosure belongs to the technical field of polymer materials, and particularly relates to a polyurea acrylate oligomer containing protected groups and a preparation method and an application thereof.

### BACKGROUND

Photo curing typically utilizes visible or ultraviolet light as a light source to rapidly transform liquid photocurable resins from a liquid state to a crosslinked material through free radical or cationic polymerization. This process is characterized by high efficiency, wide applicability, and excellent environmental performance. These features have garnered widespread attention and application of photo curing technology in fields such as coatings, inks, adhesives, photoresists, and 3D printing. Based on the chemical composition, the photo-curing resin mainly includes several components such as oligomer, active diluent, photoinitiator and light absorber. In particular, the oligomer is the main factor in determining the performance of photo-curing resin materials. Oligomers include polyester acrylates, polyurethane/polyurea acrylates, epoxy acrylates and so on, and may be divided into direct photo curing and photo curing with post-treatment according to the curing method. Usually, the network of polymers obtained by direct photo curing shows high cross-linking density and poor mechanical properties, specifically manifested as brittle and poor toughness. The network structure of the polymer may be adjusted using a post-treatment process after photo curing, thus improving the mechanical properties of the material. The Chinese patent application 202110870234.1 discloses a series of urethane acrylate oligomer, and the urethane acrylate oligomer, after photo curing, will undergo dissociation of the hindered urea bond when heated, and the exposed isocyanate group reacts with water, the urea bond, or the characteristic functional group containing active hydrogen on the side chain, so that the material's network structure features such as molecular structure and cross-link density will be changed, and ultimately the material's mechanical properties will be changed and the toughness will be greatly improved.

The photosensitive resin disclosed in the above patent realizes changes in material properties by triggering changes in the network structure by means of heating. However, these photosensitive resins are prone to gel/cross-linking phenomenon due to changes in external ambient temperature during synthesis, storage and printing, where the large site-resistant hindered urea bond dissociates and reacts with the exposed active hydrogen, bringing about resin stability problems and causing great losses and product unreliability for large-scale industrialized applications.

### SUMMARY

In order to solve the above technical problems, the present disclosure proposes a polyurea acrylate oligomer containing protected groups and a preparation method and application thereof. According to the present disclosure, protected groups are introduced in the process of synthesizing the polyurea acrylate oligomer, which is ensured not to participate in the reaction during the synthesis process, and it is convenient to introduce some highly reactive active hydrogen groups (e.g., amino group, hydroxyl group, etc.), and meanwhile to ensure the storage stability of the resin after the synthesis; and after photo curing, the protected groups may be deprotected by external stimulation, thus exposing the active hydrogen and attacking the hindered urea bonds in the main chain, which makes the network cross-linking structure and the thermodynamic properties of the material essentially changed.

In order to achieve the above objectives, the present disclosure provides the following technical solutions.

One of the technical solutions of the present disclosure:
a polyurea acrylate oligomer containing protected groups with the following structural formula:

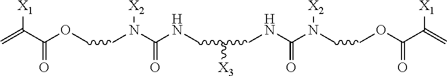

where X₁ is hydrogen or methyl;
X₂ is tert-butyl, isopropyl, piperidine or pyrazole;
X₃ is a protected group on a side chain, and the protected group is capable of dissociating to generate a characteristic groups (including hydroxyl group, amino group, carboxyl group, and so on) containing active hydrogen under conditions of heating, lighting, changing humidity, or changing acid or base, where the hydroxyl group may be protected by a formation of ketal, acetal, the amino group may be protected by a formation of tert-butyl carbamate, imine, and the carboxyl group may be protected by a formation of tert-butyl ester, o-nitrobenzene; and the X₃ includes

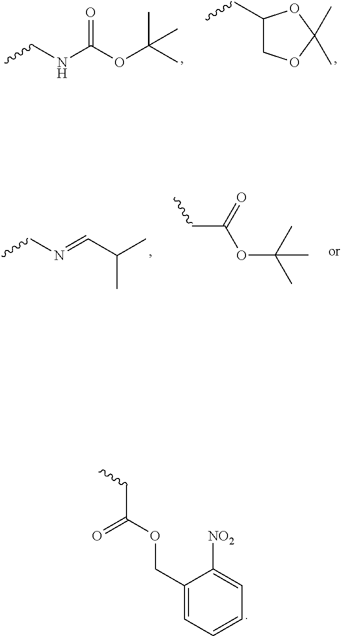

In the present disclosure, a protected characteristic group X₃ is introduced into the side chain of the polyurea acrylate oligomer, and the protected characteristic group X₃ is capable of dissociating to generate hydroxyl, amino, carboxyl, thiol, and other characteristic groups containing active hydrogen, and the dissociation of the active hydrogen may be controlled by different conditions, thus better ensuring the stability of the resin in the process of synthesizing, storing and printing. Briefly, the present disclosure protects active hydrogen and then releases it on demand by applying a certain external stimulus as needed, which will further expand the application range of photo-curing technology by enhancing the thermodynamic properties of photo-curing printing materials while ensuring high selectivity during resin synthesis and the stability of storage.

It should be noted that [symbol] in the above structural formula represents not a carbon chain, and its specific structure depends on different monomer raw materials, i.e., it represents the polyurethane prepolymer formed by diols containing X₃ groups, common diols and isocyanates. Optionally, the polyurea acrylate oligomer containing protected groups includes the following structure:

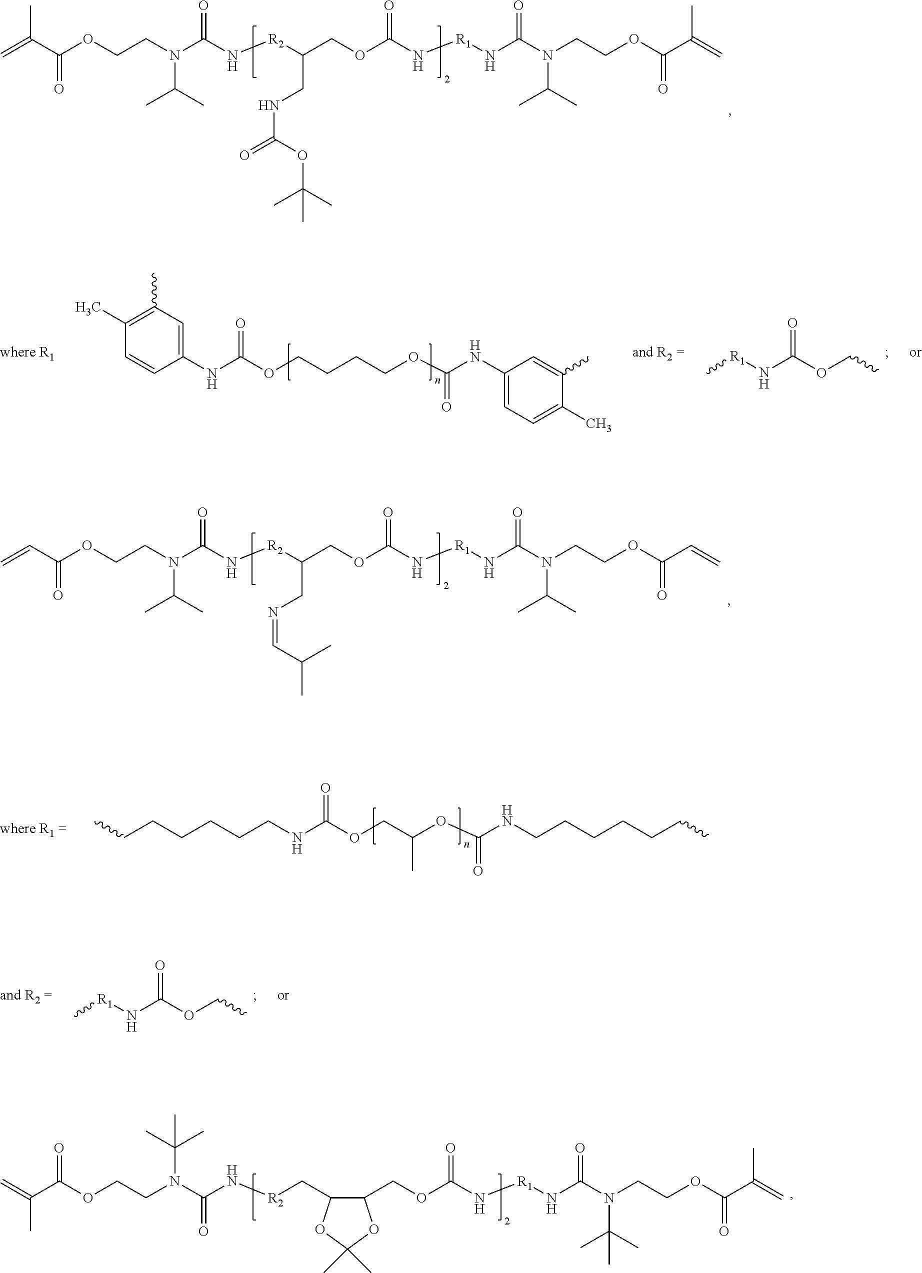

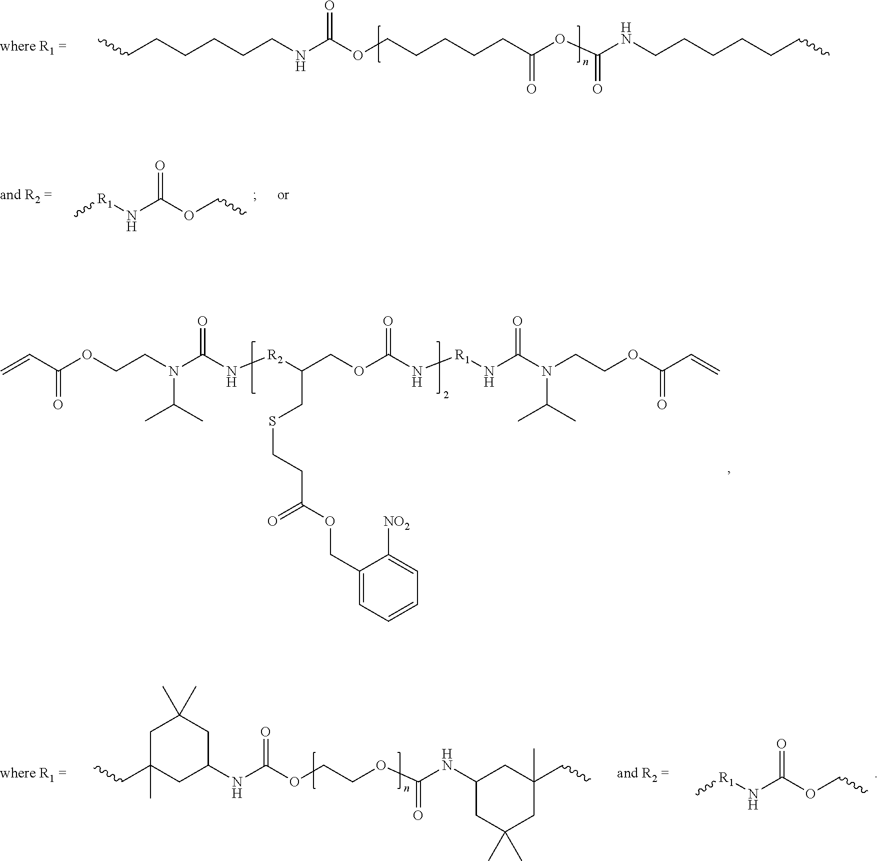

Another technical scheme of the present disclosure:
the present disclosure also provides a method of preparing the polyurea acrylate oligomer containing protected groups, including the following steps:
mixing diisocyanate, diols containing X₃ groups and common diols, and reacting at a temperature ranging from room temperature to 80 degrees Celsius (C) for 1-6 hours (h) to generate polyurethane prepolymer, confirming an isocyanate group (NCO) value of a system by a di-n-butylamine method to determine an end point of a reaction, where the diols are mainly used to modulate the molecular weight and molecular structure of the polyurethane prepolymer obtained in a first step (the wavy line portion of the resulting product is the polyurethane prepolymer formed from diols containing X₃ groups, common diols and isocyanates), and a reaction route is as follows:

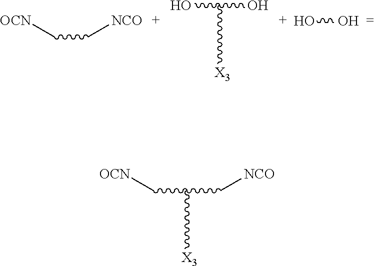

mixing the polyurethane prepolymer with a hindered amine acrylate

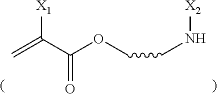

and allowing for reacting at 50 to 100° C. for 1 to 6 h to generate the polyurea acrylate oligomer containing protected groups by a following reaction route:

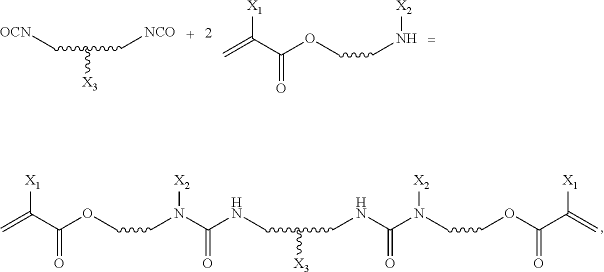

where the diols containing X₃ groups include one of tert-butyl N-(2,3-dihydroxypropyl)carbamate

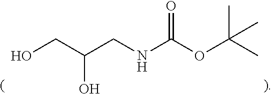

N-(2,3-dihydroxypropyl)isobutyraldehyde imine

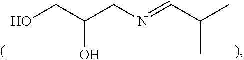

1,1-dimethyl-3-[(2,3-dihydroxypropyl)thio] ethyl propionate

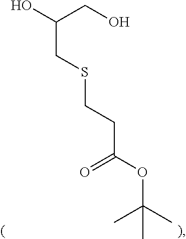

### (+)-2,3-O-isopropylidene-L-threitol

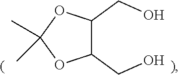

and 3-[(2,3-dihydroxypropyl)sulfenyl] o-nitrophenethyl ester

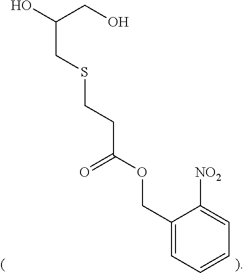 [SMILES: O=C(CCSCC(O)CO)OCc1ccccc1[N+](=O)[O-]]

Optionally, a molar ratio of the diols containing the X₃ groups to the common diol is 1:(0-10) and a number of moles of the diols is not 0; and

a molar ratio of the diisocyanate to a total active hydrogen in the diols containing the X₃ groups and in common diols is (1.1 to 10): 1.

Optionally, the diisocyanate includes one of hexamethylene diisocyanate, toluene diisocyanate, p-toluene diisocyanate, 4,4′-diphenylmethane diisocyanate, isophorone diisocyanate and 1,5-naphthalene diisocyanate;
the common diols include one of polyethylene glycol, polypropylene glycol, polytetramethylene ether glycol, polycaprolactone and polydiethylene glycol adipate; and
the hindered amine acrylate includes one of tert-butylaminoethyl methacrylate, tert-butylaminoethyl acrylate, isopropylaminoethyl methacrylate, isopropylaminoethyl acrylate, piperidinylaminoethyl acrylate or piperidinylaminoethyl methacrylate.

Another technical scheme of the present disclosure:
the present disclosure also provides a photo-curing resin prepared from the polyurea acrylate oligomer containing protected groups. The photo-curing resin prepared by the present disclosure may be used as a 3D printing material.

Another technical scheme of the present disclosure:
the present disclosure also provides a method of preparing the photo-curing resin, where the polyurea acrylate oligomer containing protected groups is mixed with an active diluent, and then a photoinitiator and an inhibitor are added to obtain a mixture, and the mixture is subjected to photo curing, and remove the protected groups, and then treated for 1-10 h at 50-140° C. to obtain the photo-curing resin.

Optionally, a method of removing the protected groups includes heating, lighting, humidity changing or acid-base changing. Different protected groups are removed in different ways, and reaction routes are as follows:

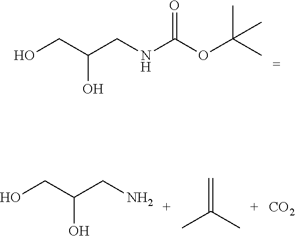

(reaction under acidic conditions);

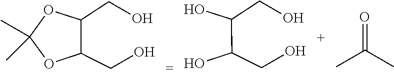

(reaction with presence of acid and water, under heated conditions);

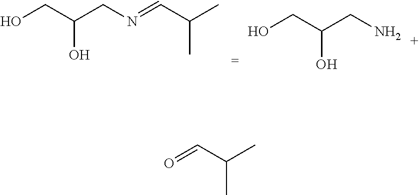

(reaction under certain humidity conditions);

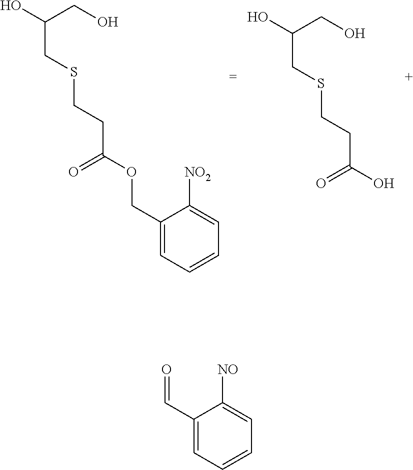

(reaction under ultraviolet irradiation); and

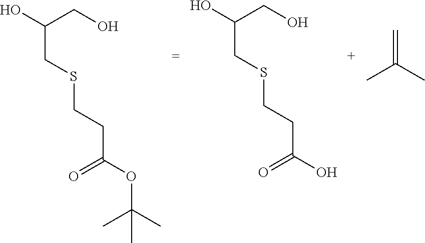

(reaction under acidic and heated conditions).

Acidic conditions are required to remove the isobutylene group from the tert-butyl carbamate group so as to obtain the amino group; a certain degree of humidity is required to remove the aldehyde group from the imine group so as to obtain the amino group; the ketal structure requires heating in the presence of acid and water to remove acetone and obtain the hydroxyl group; the isobutylene group may be removed from the tert-butyl ester group to yield a carboxyl group under acidic and heated conditions; and the nitrobenzaldehyde may be removed from the o-nitrophenyl ester group to give the carboxyl group under ultraviolet (UV) light.

Optionally, a weight ratio of the active diluent to the polyurea acrylate oligomer containing protected groups is (0.05 to 0.5): 1, more optionally (0.2 to 0.4): 1;
a weight ratio of the photoinitiator to the polyurea acrylate oligomer containing protected groups is (0.01 to 0.05): 1, more optionally 0.01:1;
a weight ratio of the inhibitor to the polyurea acrylate oligomer containing protected groups is (0.0005 to 0.002): 1, more optionally 0.002:1.

The active diluent is used to regulate the resin system in terms of viscosity and the mechanical properties of sample after the curing of resin, and the photoinitiator and the inhibitor regulate a photopolymerization reaction in terms of kinetics.

The active diluent is a low-viscosity mono- or multifunctional acrylate or methacrylate compound, e.g., the active diluent is tetrahydrofuran acrylate, isobornyl acrylate, 2-phenoxyethyl acrylate, or isooctyl acrylate.

In the present disclosure, there is no special limitation on the types of photoinitiators and inhibitor, and it is sufficient to use photoinitiators and inhibitor that are common in the field, for example, photoinitiator 819 and inhibitor p-hydroxyanisole.

The light source used is compatible with the photoinitiator and may be a UV or visible light source.

The polyurea acrylate oligomer containing protected groups are photoinitiated polymerized to form a crosslinked network, and then processed under certain conditions (heating, lighting, humidity changing or acid-base changing) to remove the protected groups and expose the active hydrogen groups, and then placed at a certain temperature, the hindered urea bonds in the network dissociate, reacting with the active hydrogen groups on the side chains to obtain a new polymer network. On the one hand, the introduction of protective groups ensures the stability of the resin in the process of synthesis, application and storage; on the other hand, the subsequent deprotection gives access to active hydrogen groups, which react with the hindered urea bonds in the network to form a polyacrylate-polyurethane crosslinked network, resulting in significant changes in the mechanical properties of the samples compared to those prior to treatment with heating, lighting, humidity changing, or acid/base changing, with improved toughness.

Another technical scheme of the present disclosure:

The present disclosure also provides an application of the polyurea acrylate oligomer containing protected groups in preparing photo-curing resins.

The photo-curing resins prepared using the polyurea acrylate oligomer containing protected groups of the present disclosure have good storage stability, with viscosity remaining essentially unchanged after one month of placement.

Compared with the prior art, the present disclosure has the following advantages and technical effects.

The present disclosure introduces a protective group in the polyurea acrylate oligomer, which ensures the stability of the resin during synthesis, storage, and printing;

The protective groups in the polyurea acrylate oligomer of the present disclosure may be deprotected under certain conditions (heating, lighting, humidity changing or acid-base changing), and the properties of the material may be regulated and improved by reacting with the dynamic bonds in the network.

### BRIEF DESCRIPTION OF THE DRAWINGS

The accompanying drawings, which form part of this application, are used to provide a further understanding of this application, and the schematic embodiments of this application and their description are used to explain this application and do not constitute an undue limitation of this application. In the accompanying drawings:

FIG. 1 shows the results of viscosity changes at different times for the polyurea acrylate oligomer containing protected groups in Embodiment 1;

FIG. 2 shows the results of viscosity changes at different times for the polyurea acrylate oligomer containing protected groups in Embodiment 2;

FIG. 3 shows the results of viscosity change at different times for the polyurea acrylate oligomer containing protected groups in Embodiment 3;

FIG. 4 shows the results of viscosity change at different times for the polyurea acrylate oligomer containing protected groups in Embodiment 4;

FIG. 5 shows the results of viscosity change at different times for the polyurea acrylate oligomer in Comparative embodiment 1 which does not contain protected groups.

### DETAILED DESCRIPTION OF THE EMBODIMENTS

Various exemplary embodiments of the present disclosure are now described in detail, which detailed description should not be considered as a limitation of the present disclosure, but rather should be understood as a more detailed description of certain aspects, features and embodiments of the present disclosure.

It is to be understood that the terms described in the present disclosure are only intended to describe particular embodiments and are not intended to limit the present disclosure. Further, for the range of values in the present disclosure, it should be understood that each intermediate value between the upper and lower limits of the range is also specifically disclosed. Each smaller range between any stated value or intermediate value within the stated range and any other stated value or intermediate value within the stated range is also included within the present disclosure. The upper and lower limits of these smaller ranges may be independently included or excluded from the range.

Unless otherwise indicated, all technical and scientific terms used herein have the same meaning as is commonly understood by those of ordinary skill in the art described in the present disclosure. While only preferred methods and materials are described herein, any methods and materials similar or equivalent to those described herein may also be used in the implementation or testing of the present disclosure. All literature referred to in this specification is incorporated by reference for the purpose of disclosing and describing the methods and/or materials associated with the literature. In the event of a conflict with any incorporated literature, the contents of this specification shall prevail.

Various improvements and variations may be made to specific embodiments of the specification of the present disclosure without departing from the scope or spirit of the present disclosure, as will be apparent to those skilled in the art. Other embodiments obtained from the specification of the present disclosure will be apparent to those skilled in the art. The specification and embodiments of the present disclosure are exemplary only.

As used herein, the terms “contain”, “include”, “have”, “comprise”, and so on, are open-ended terms, i.e., meant to include but not be limited to.

The respective raw materials used in the embodiments of the present invention are obtained through commercially available purchases. By way of example, all of the ingredients used in this present disclosure are purchased from Aladdin, where the CAS number of polydiethylene glycol adipate is: 24938-37-2.

The technical schemes of the present disclosure are further described below by means of embodiments.

### Embodiment 1

(1) 0.5 mol of polytetramethylene ether glycol (PTMEG, M_{w}=1000) and 1 mol of p-toluene diisocyanate (TDI, and 0.3 M_{w}=174), mol of tert-butyl N-(2,3-dihydroxypropyl)carbamate are added to the reactor, and the reaction is carried out for 4 h at a controlled temperature of 50° C., the reaction endpoints are determined by confirming the NCO value of the system by the di-n-butylamine method, and the polyurethane prepolymer is obtained;

(2) the isopropylaminoethyl methacrylate of 0.4 mol is added dropwise to the reactor through the constant pressure funnel, and the temperature is controlled to be 80° C. during the dropwise addition, and after the dropwise addition is completed, the reaction is kept warm and continued for 4 h, and the endpoint of the reaction is judged by the NCO value of the system through the di-n-butylamine method, and the polyurea acrylate oligomer containing protected groups

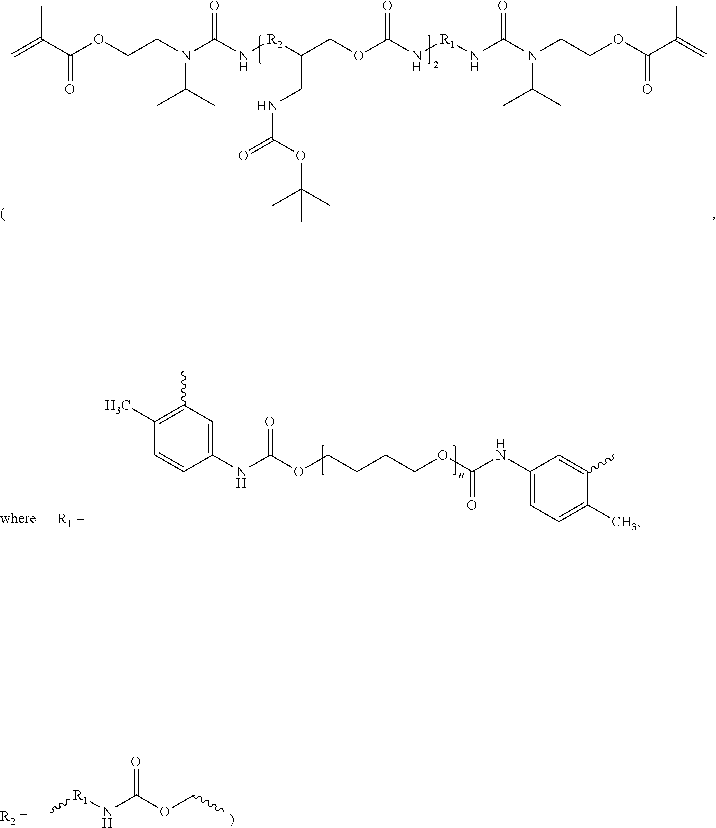

is obtained.

The above polyurea acrylate oligomer containing protected groups is taken as 10 grams (g) and 3 g of active diluent tetrahydrofuran acrylate is added, 0.1 g of photoinitiator 819 and 0.02 g of polymerization inhibitor p-hydroxyanisole are added to obtain the liquid photo-curing resin precursor, and the light source is a high-pressure mercury lamp, and the sample strips conforming to the standard of ASTM D412 are casted in a transparent mold for testing the unidirectional tensile properties; the sample strips are soaked in a hydrochloric acid solution for 10 h to remove the protected groups, and then placed in the oven at 120° C. for treatment of 6 h to test the unidirectional tensile properties. The sample strip before treatment (i.e., directly cast out material) conforming to ASTM D412 has an elongation at break of 100% and a breaking strength of 3 megapascal (MPa); the sample strip after treatment (i.e., material that has undergone removal of the protective groups) conforming to ASTM D412 has an elongation at break of 600% and a breaking strength of 30 MPa.

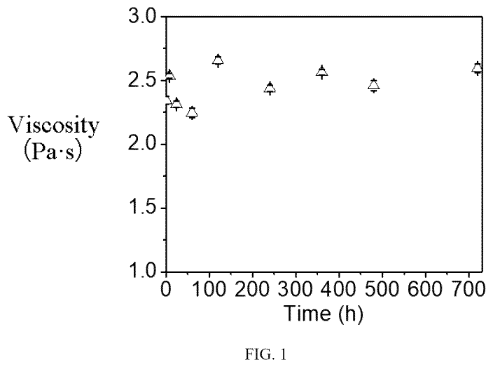

The change in viscosity of the liquid photo-curing resin precursor for different times of placement at 50° C. is measured by means of a viscometer. As shown in FIG. 1, the viscosity remains essentially unchanged after one month of placement.

### Embodiment 2

(1) 0.5 mol of polypropylene glycol (PPG, M_{w}=600) and 1 mol of hexamethylene diisocyanate (HDI, M_{w}=168), and 0.25 mol of N-(2,3-dihydroxypropyl)isobutyraldehyde imine are added to the reactor at a controlled temperature of 50° C., and the end point of the reaction is determined by confirming the NCO value of the system by the di-n-butylamine method, and then the polyurethane prepolymer is obtained;

(2) the isopropylaminoethyl acrylate of 0.5 mol is added dropwise to the reactor through the constant pressure funnel, and the temperature is controlled to be 50° C. during the dropwise addition, and after the dropwise addition is completed, the reaction is kept warm and continued, and the endpoint of the reaction is judged by the NCO value of the system through the di-n-butylamine method, and the polyurea acrylate oligomer containing protected groups

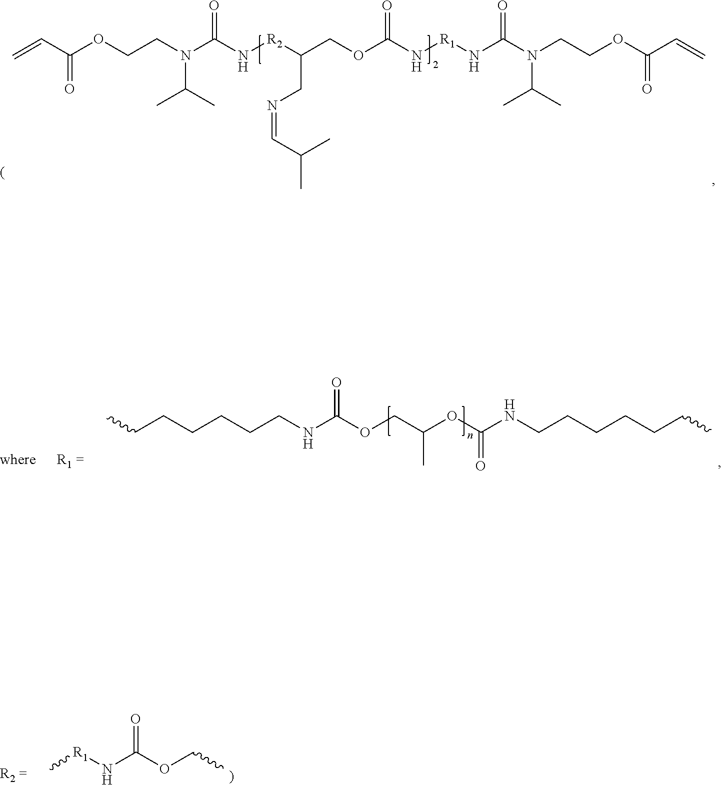

is obtained.

The above polyurea acrylate oligomer containing protected groups is taken as 10 g and 2 g of active diluent isobornyl acrylate is added, 0.1 g of photoinitiator 819 and 0.02 g of polymerization inhibitor p-hydroxyanisole are added to obtain the liquid photo-curing resin precursor, and the light source is a high-pressure mercury lamp, and the sample strips conforming to the standard of ASTM D412 are casted in a transparent mold for testing the unidirectional tensile properties; the above sample strips are treated in constant temperature and humidity with an air humidity of 50% or more for 24 h, and then placed under an oven at 70° C. for 6 h to test the unidirectional tensile properties. The sample strip before treatment has an elongation at break of 200% and a breaking strength of 12 MPa; the sample strip after treatment has an elongation at break of 500% and a breaking strength of 40 MPa.

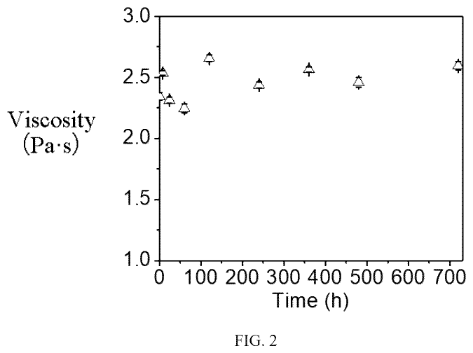

The change in viscosity of the liquid photo-curing resin precursor for different times of placement at 50° C. is measured by means of a viscometer. As shown in FIG. 2, the viscosity remains essentially unchanged after one month of placement.

### Embodiment 3

(1) 0.5 mol of polycaprolactone (PCL, M_{w}=1500) and 1 mol of hexamethylene diisocyanate (HDI, M_{w}=168), and 0.25 mol of (+)-2,3-O-isopropylidene-L-threitol are added to the reactor at a controlled temperature of 50° C., and the end point of the reaction is determined by confirming the NCO value of the system by the di-n-butylamine method, and then the polyurethane prepolymer is obtained;

(2) the tert-butylaminoethyl methacrylate of 0.5 mol is added dropwise to the reactor through the constant pressure funnel, and the temperature is controlled to be 50° C. during the dropwise addition, and after the dropwise addition is completed, the reaction is kept warm and continued, and the endpoint of the reaction is judged by the NCO value of the system through the di-n-butylamine method, and the polyurea acrylate oligomer containing protected groups

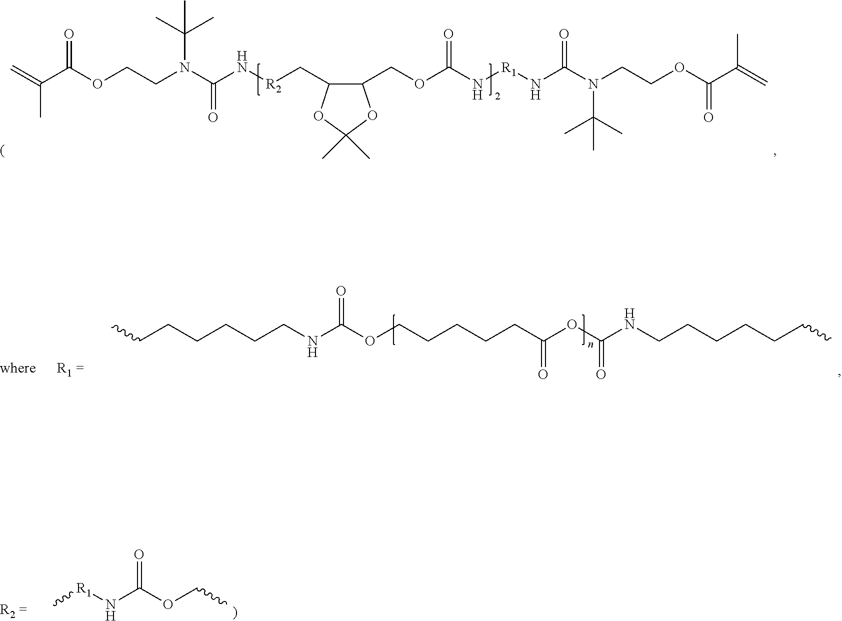

is obtained.

The above polyurea acrylate oligomer containing protected groups is taken as 10 g and 2 g of active diluent 2-phenoxyethyl acrylate is added, 0.1 g of photoinitiator 819 and 0.02 g of polymerization inhibitor p-hydroxyanisole are added to obtain the liquid photo-curing resin precursor, and the light source is a high-pressure mercury lamp, and the sample strips conforming to the standard of ASTM D412 are casted in a transparent mold for testing the unidirectional tensile properties; the above sample strips are immersed in a weak acidic solution and then placed in a constant temperature and humidity with an air humidity of 50% or more and a temperature of 60° C. for 1 h, and then transferred to an oven at 80° C. for 6 h to test the unidirectional tensile properties. The sample strip before treatment has an elongation at break of 350% and a breaking strength of 8 MPa; the sample strip after treatment has an elongation at break of 1000% and a breaking strength of 40 MPa.

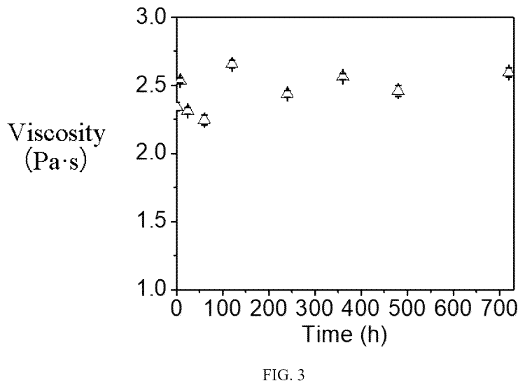

The change in viscosity of the liquid photo-curing resin precursor for different times of placement at 50° C. is measured by means of a viscometer. As shown in FIG. 3, the viscosity remains essentially unchanged after one month of placement.

### Embodiment 4

(1) 0.5 mol of polyethylene glycol (PEG, M_{w}=2000) and 1 mol of isophorone diisocyanate (IPDI, M_{w}=222), and 0.25 mol of 3-[(2,3-dihydroxypropyl)sulfenyl]o-nitrophenethyl ester are added to the reactor at a controlled temperature of 50° C., and the end point of the reaction is determined by confirming the NCO value of the system by the di-n-butylamine method, and then the polyurethane prepolymer is obtained;

(2) the isopropylaminoethyl acrylate of 0.5 mol is added dropwise to the reactor through the constant pressure funnel, and the temperature is controlled to be 50° C. during the dropwise addition, and after the dropwise addition is completed, the reaction is kept warm and continued, and the endpoint of the reaction is judged by the NCO value of the system through the di-n-butylamine method, and the polyurea acrylate oligomer containing protected groups

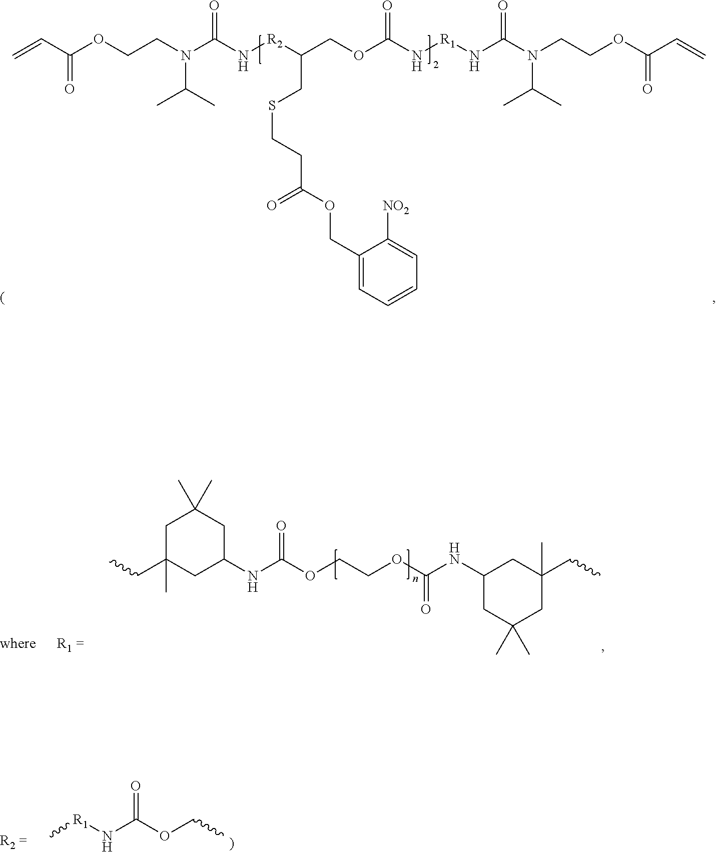

is obtained.

The above polyurea acrylate oligomer containing protected groups is taken as 10 g and 2 g of active diluent isooctyl acrylate is added, 0.1 g of photoinitiator 819 and 0.02 g of polymerization inhibitor p-hydroxyanisole are added to obtain the liquid photo-curing resin precursor, and the light source is a high-pressure mercury lamp, and the sample strips conforming to the standard of ASTM D412 are casted in a transparent mold for testing the unidirectional tensile properties; the above sample strips are treated with light in a UV curing phase for 0.5 h and then placed under an oven at 100° C. for 6 h to test the unidirectional tensile properties. The sample strip before treatment has an elongation at break of 100% and a breaking strength of 4 MPa; the sample strip after treatment has an elongation at break of 600% and a breaking strength of 30 MPa.

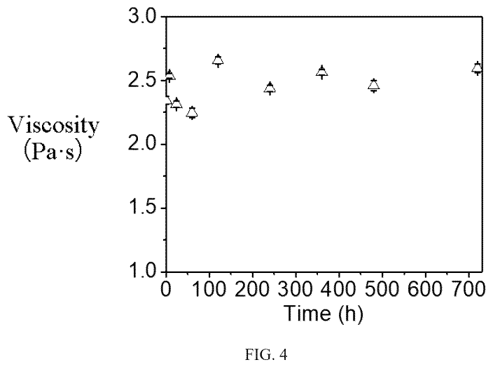

The change in viscosity of the liquid photo-curing resin precursor for different times of placement at 50° C. is measured by means of a viscometer. As shown in FIG. 4, the viscosity remains essentially unchanged after one month of placement.

### Comparative Embodiment 1 (Unprotected Groups)

(1) 0.5 mol of polyethylene glycol (PEG, M_{w}=2000) and 1 mol of isophorone diisocyanate (IPDI, M_{w}=222), and 0.25 mol of 3-[(2,3-Dihydroxypropyl)sulfenyl] propanoic acid are added to the reactor at a controlled temperature of 50° C., and the end point of the reaction is determined by titration to obtain the polyurethane prepolymer;

(2) the isopropylaminoethyl acrylate of 0.5 mol is added dropwise to the reactor through the constant pressure funnel, and the temperature is controlled to be 50° C. during the dropwise addition, and after the dropwise addition is completed, the reaction is kept warm and continued, and the endpoint of the reaction is judged by the NCO value of the system through the di-n-butylamine method, and the polyurea acrylate oligomer containing carboxyl groups (unprotected)

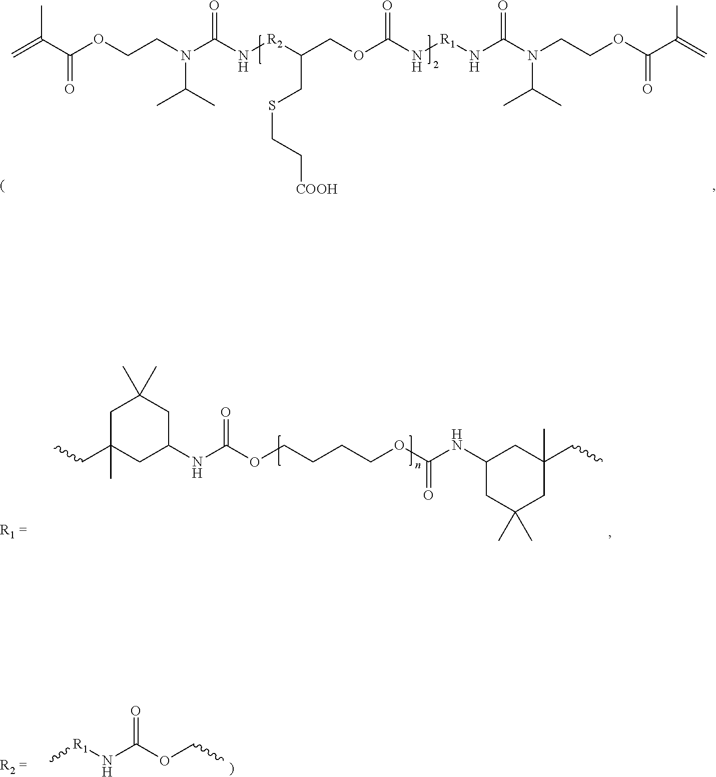

is obtained.

The above polyurea acrylate oligomer containing carboxyl groups is taken as 10 g, and 4 g of active diluent isooctyl acrylate, 0.1 g of photoinitiator 819, and 0.02 g of polymerization inhibitor p-hydroxyanisole are added, and the liquid photo-curing resin precursor is obtained.

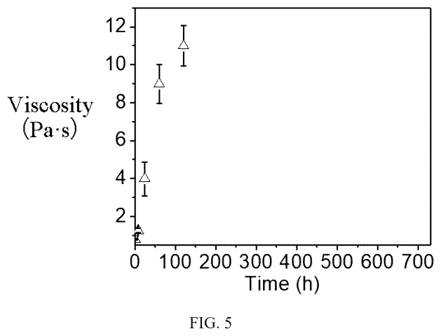

The change in viscosity of the liquid photo-curing resin precursor for different times of placement at 50° C. is measured by means of a viscometer. As shown in FIG. 5, the viscosity changes significantly after one day of placement, and the resin gels after five days (the viscosity is not measurable).

As may be seen from Embodiment 4 and Comparative embodiment 1, the present disclosure substantially improves the storage stability of the resin by introducing protective groups.

The above are only preferable specific embodiments of the present application, but the scope of protection of the present application is not limited thereto, and any changes or substitutions that are readily conceivable by any person skilled in the art within the technical scope disclosed in the present application should be covered by the scope of protection of the present application. Therefore, the scope of protection of this application should be based on the scope of protection of the claims.

What is claimed is:

1. A polyurea acrylate oligomer containing protected groups X3, comprising a following structural formula: 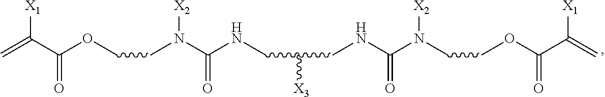
wherein X₁ is hydrogen or methyl;
X₂ is tert-butyl, isopropyl, piperidine or pyrazole; and
X₃ is

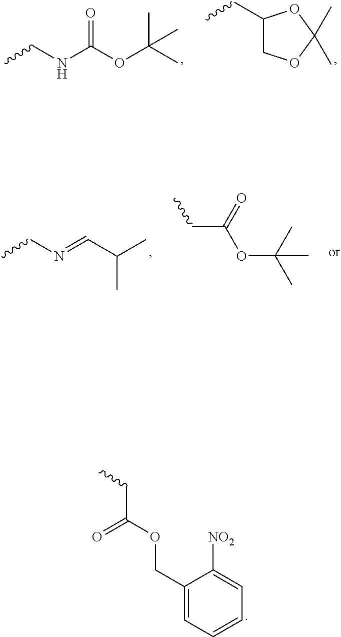

2. The polyurea acrylate oligomer containing protected groups according to claim 1, wherein following structure is comprised:

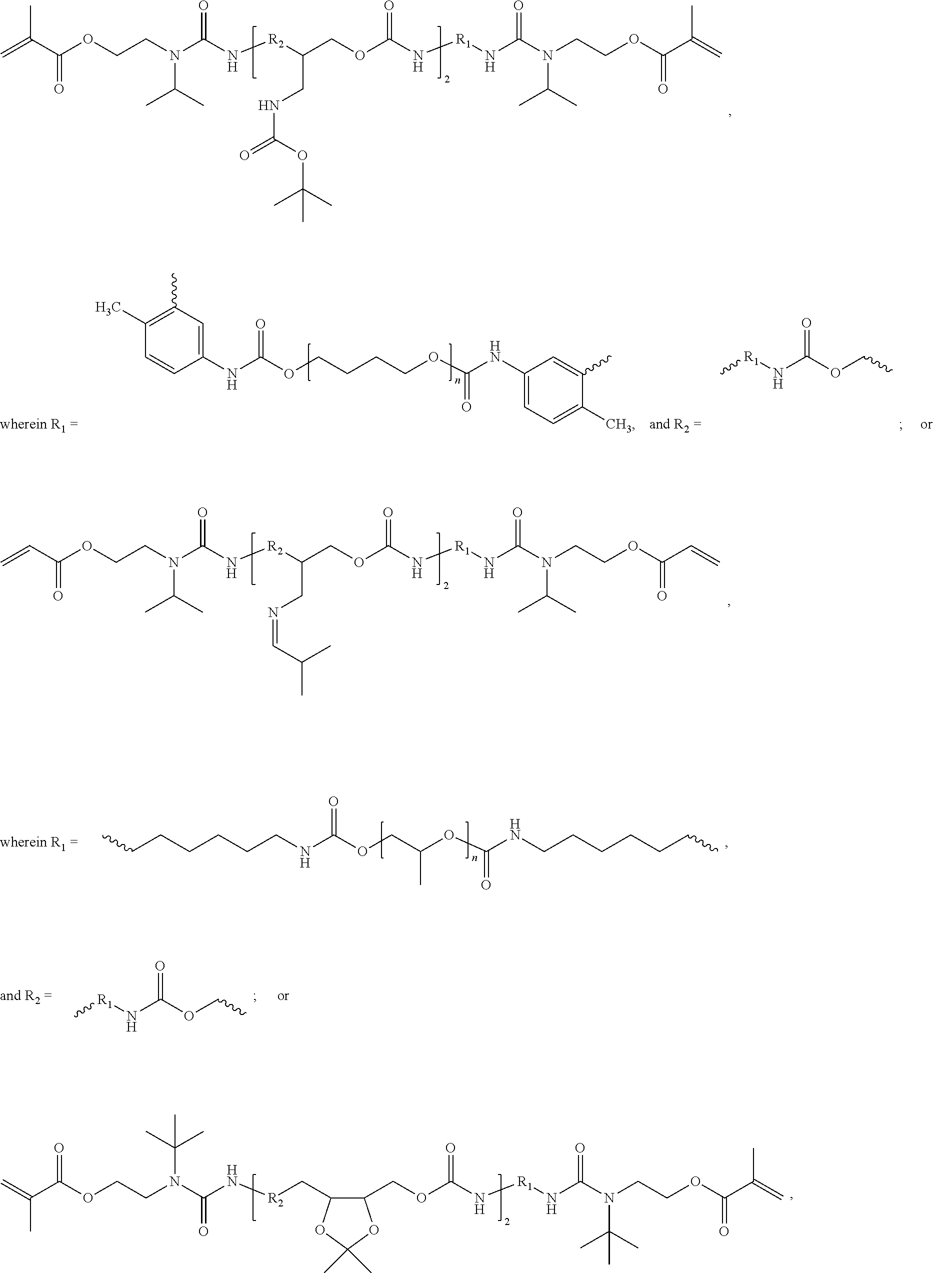

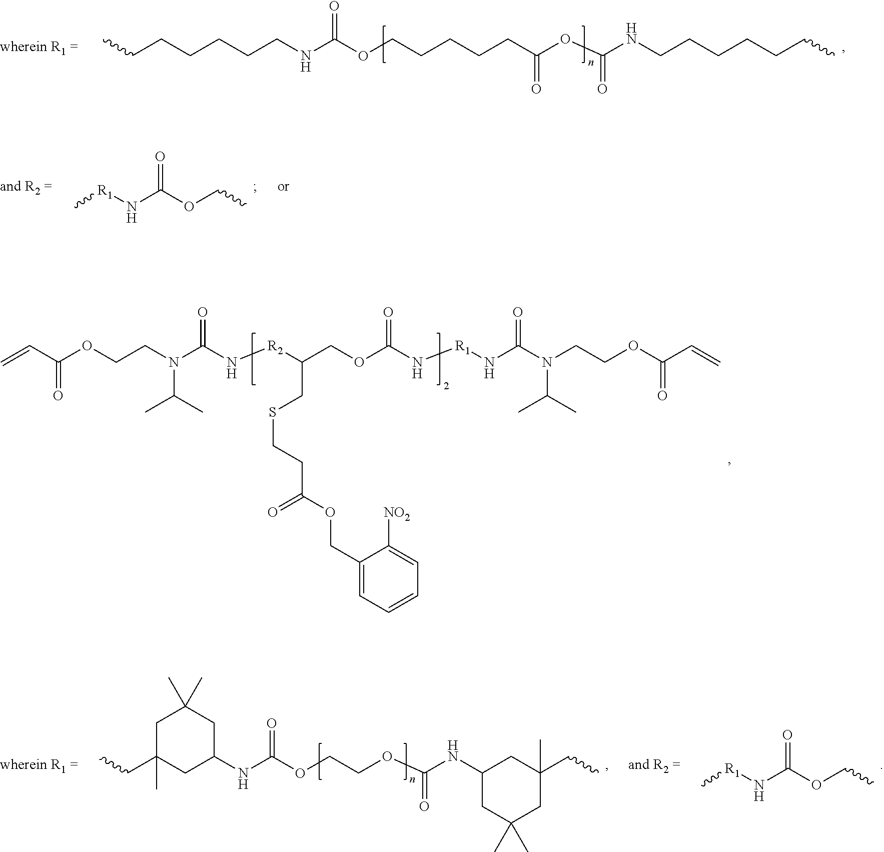

3. A method of preparing the polyurea acrylate oligomer containing protected groups according to claim 1, comprising following steps:
mixing diisocyanate, diols containing X₃ groups and common diols, and reacting at a temperature ranging from room temperature to 80 degrees Celsius for 1-6 hours to generate polyurethane prepolymer;
mixing the polyurethane prepolymer with a hindered amine acrylate and allowing for reacting at 50 to 100 degrees Celsius for 1 to 6 hours to generate the polyurea acrylate oligomer containing protected groups; wherein
the diols containing X₃ groups comprises one of tert-butyl N-(2,3-dihydroxypropyl)carbamate, N-(2,3-dihydroxypropyl)isobutyraldehyde imine, 1,1-dimethyl-3-[(2,3-dihydroxypropyl)thio]ethyl propionate, (+)-2,3-O-isopropylidene-L-threitol, and 3-[(2,3-dihydroxypropyl)sulfenyl]o-nitrophenethyl ester.

4. The method of preparing the polyurea acrylate oligomer containing protected groups according to claim 3, wherein a molar ratio of the diols containing the X₃ groups to the common diol is 1:(0-10) and a number of moles of the diols is not 0; and
a molar ratio of the diisocyanate to a total active hydrogen in the diols containing the X₃ functional monomers and in common diols is (1.1 to 10):1.

5. The method of preparing the polyurea acrylate oligomer containing protected groups according to claim 4, wherein the diisocyanate comprises one of hexamethylene diisocyanate, p-toluene diisocyanate, 4,4′-diphenylmethane diisocyanate, isophorone diisocyanate and 1,5-naphthalene diisocyanate;
the common diols comprise one of polyethylene glycol, polypropylene glycol, polytetramethylene ether glycol, polycaprolactone and polydiethylene glycol adipate; and
the hindered amine acrylate comprises one of tert-butylaminoethyl methacrylate, tert-butylaminoethyl acrylate, isopropylaminoethyl methacrylate, isopropylaminoethyl acrylate, piperidinylaminoethyl acrylate or piperidinylaminoethyl methacrylate.

6. A photo-curing resin prepared by the polyurea acrylate oligomer containing protected groups according to claim 1.

7. A method of preparing the photo-curing resin according to claim 6, comprising mixing the polyurea acrylate oligomer containing protected groups with an active diluent, and then a adding a photoinitiator and an inhibitor to obtain a mixture, and subjecting the mixture to photo curing to remove the protected groups, and then treating for 1-10 hours at 50-140 degrees Celsius to obtain the photo-curing resin.

8. The method of preparing the photo-curing resin according to claim 7, wherein a weight ratio of the active diluent to the polyurea acrylate oligomer containing protected groups is (0.05 to 0.5):1;
a weight ratio of the photoinitiator to the polyurea acrylate oligomer containing protected groups is (0.01 to 0.05):1; and
a weight ratio of the inhibitor to the polyurea acrylate oligomer containing protected groups is (0.0005 to 0.002):1.

9. An application of the polyurea acrylate oligomer containing protected groups according to claim 1 in preparing photo-curing resins.
